(19) United States
(12) Patent Application Publication
(10) Pub. No.: US 2024/0352411 A1
(43) Pub. Date: Oct. 24, 2024

(54) ANIMAL-FREE MATERIALS FOR CELL CULTIVATION IN BIOREACTORS AND METHODS OF MAKING AND USING THEREOF

(71) Applicant: NouBio Inc., Dover, DE (US)

(72) Inventors: Samad Ahadian, Dover, DE (US); Stephen G. Gray, Co. Louth (IE)

(73) Assignee: NouBio Inc., Dover, DE (US)

(21) Appl. No.: 18/144,264

(22) Filed: May 8, 2023

Related U.S. Application Data
(60) Provisional application No. 63/460,624, filed on Apr. 20, 2023.

(51) Int. Cl. C12N 5/00; A23J 3/22

(52) U.S. Cl. CPC C12N 5/0075; A23J 3/227; C12N 2531/00; C12N 2533/50; C12N 2533/70

(57) ABSTRACT

Microstructures made of dairy products derived from fermentation or mammalian cells as edible or biodegradable microcarriers for cell culture in bioreactors for biomedical applications ranging from tissue engineering, cell-based foods or materials (e.g., cultivated meat, leather), vaccine production, human tissue fabrication, and cell/gene therapy. Edible or biodegradable microcarriers have the potential to be used for the scalable growth of cells in bioreactors in animal serum-free media without the need to be separated from the microcarriers for a final cell-based meat or other cell-based products. Edible microcarriers can act as a replacement for fat in various products and can be modified with the inclusion of flavor molecules and custom proteins and extracellular matrix components to enhance the final creation of cell-based meat or other cell-based products in a bioreactor.

### CROSS-REFERENCE TO RELATED APPLICATIONS SECTION

This application claims priority to U.S. Application Ser. No. 63/460,624 filed on Apr. 20, 2023, the entire content is hereby fully incorporated by reference in their entirety.

### FIELD OF THE INVENTION

The present invention relates to the use of microstructures made of dairy products derived from fermentation or mammalian cells as edible or biodegradable microcarriers for cell culture in bioreactors for biomedical applications ranging from tissue engineering, cell-based foods or materials (e.g., cultivated meat, leather), vaccine production, human tissue fabrication, and cell/gene therapy.

### BACKGROUND OF THE INVENTION

Microcarriers are small particles that are used in dynamic cell culture systems to support the growth of adherent cells. These particles can be made from various materials, such as polystyrene, glass, or silicone, and can be coated with different substances to alter their surface properties or made with different levels of porosity. Microcarriers are commonly used in biotechnology and pharmaceutical manufacturing for biomedical applications, such as vaccine production and cell population expansion. However, current commercial microcarriers have the disadvantage of requiring cells to be detached from their surface before they can be used in downstream applications, which can be a limiting factor in some situations. Some animal-derived materials are used to make or coat those microcarriers. In addition, the majority of microcarrier technologies or materials cannot be modified or enhanced for flavor, texture, cell density or the protection of cells against shear stress damage in bioreactors. Therefore, there is a need for improved microcarriers that address these issues. It is with these limitations in mind that the present invention was made.

### SUMMARY OF THE INVENTION

The present invention relates to the use of microstructures made of dairy products derived from fermentation or mammalian cells as edible or biodegradable microcarriers for cell culture in bioreactors for biomedical applications ranging from tissue engineering, cell-based foods or materials (e.g., cultivated meat, leather), vaccine production, human tissue fabrication, and/or cell/gene therapy. In an embodiment, these edible microcarriers have the potential to be used for the scalable growth of cells in bioreactors in animal-free media without the need to be separated from the microcarriers for a final meat product. The edible microcarriers can act as a replacement for fat in various products and can be modified with the inclusion of flavor molecules and custom proteins and extracellular matrix components to enhance the final creation of meat tissues in a bioreactor. In an embodiment, the microcarriers of the present invention are edible because they are derived from or comprise components from whey (e.g., fermented whey). In an embodiment, the proteins and flavor molecules that are added to the microcarriers are also edible, examples of which are disclosed herein.

One aspect of the present invention involves the use of these edible microcarriers to grow cultivated meat in bioreactors. The bioreactors contain an animal-free media formulation that converts dairy-based compounds into recombinant proteins to match all few hundred proteins found in animal serum, allowing for the cultivation of meat tissue in a more sustainable and ethical manner. In an embodiment, the present invention also includes methods of creating these dairy-based microstructures, which can be used as scaffolds or substrates for cell culture. These microstructures offer alternative solutions to various problems in the field and can be tailored to meet the specific needs of various applications.

There are several ways in which the edible microcarriers of the present invention can be modified to enhance the final flavor or texture of the cultivated meat created in bioreactors. For example, the microcarriers can be infused with various flavor molecules, such as natural plant-based extracts or synthetic flavor compounds, to add desired tastes, textures, and aromas to the meat. In an embodiment, the microcarriers can be supplemented with custom proteins that are specifically designed to provide specific flavors and textures to the cultivated meat.

The benefits and unique features of the present invention will be further described in the following detailed description of various non-limiting embodiments, in conjunction with the accompanying protocols and FIGURES (see below). Additions to this microcarrier technology can incorporate protocols and technologies to not only enhance flavor and texture, but also increase cell density and other properties of cultivated material. For example, these additions can be included in the final process to protect cells cultured on the edible microcarriers from shear stress damage during growth in bioreactors.

### BRIEF DESCRIPTION OF THE SEVERAL VIEWS OF THE DRAWINGS

FIG. 1 shows a drawing of cell seeding on solid (A) and porous (B) microparticles to make cell culture using the microstructures of the present invention.

### DETAILED DESCRIPTION OF THE INVENTION

The present disclosure relates to dairy-based microstructures for use in various applications, including cell culture. These dairy-based microstructures can be made from various dairy-based materials, such as whey protein microparticles or whey protein sheets. In an embodiment, these dairy-based microstructures may be coated with other materials, such as proteins, peptides, or blood products, to enhance their functionality. In an embodiment, these dairy-based microstructures can also be used to culture a variety of animal and human cells. In a variation, the dairy-based microstructures may be used to produce cultivated products, such as cell-based meat. In an embodiment, these dairy-based microstructures may also be sustainably harvested, for example, from milk-producing animals raised on regenerative farms. In a variation, the present disclosure also includes methods of making and using these microstructures for cell culture and other uses, as well as kits comprising these microstructures. The kits may comprise the microstructures themselves and optionally contain media, cells, instructions and other products that are necessary to practice the present invention.

In an embodiment, aspects of the present disclosure relate to dairy-based microstructures for use in cell culture and other applications. These microstructures may be made from milk proteins such as whey or casein proteins, and can be processed into various forms, including microparticles and sheets. In a variation, these microstructures may be crosslinked or non-crosslinked, and may be produced using binding agents such as transglutaminase. These microstructures may also be coated with materials, such as cell-binding ligands, extracellular matrix products, or blood serum products, and may be covalently or non-covalently coupled to the dairy-based microstructure.

In an embodiment, these dairy-based microstructures can be used to expand cultures of various animal and human cells, including muscle cells, nerve cells, fat cells, and stem cells such as embryonic stem cells, induced pluripotent stem cells, and adult stem cells. These cells may be sourced from humans or a variety of animals, including but not limited to other primates, cows, sheep, pigs, goats, camels, horses, llamas, alpacas, crocodiles, and bison. These microstructures may also be used to produce cultivated products, such as skin, leather, suede, wool, tissues, and organs.

In an embodiment, the present disclosure also relates to the use of whey derived from fermentation or mammalian cells in the production of dairy-based microstructures for various applications, including cell culture. Other applications include using the dairy-based microstructures in the production of cell-based products, in the production of organs, or tissues, to make organlike structures or systems that can be used for drug testing, in the production of biofuels, in the production of products that can be isolated from the structurally organized grown cells, for antibody and antigen production, for proteins isolated from cells, for recombinant proteins or vesicles, or for a plurality of other purposes.

Fermented whey is produced through the fermentation of whey protein using microorganisms, such as bacteria or yeast. In some embodiments, fermented whey may be used in the production of microstructures, such as microparticles or sheets, which may optionally be crosslinked (or non-crosslinked) using binding agents, such as transglutaminase. In an embodiment, these microstructures may be coated with other biomolecules or materials, such as cell-binding ligands, extracellular matrix products, or blood serum products, and may be covalently or non-covalently coupled to the fermented whey microstructure.

In an embodiment, whey and/or whey protein may be derived from mammalian cells. Whey is the liquid portion that is present and is formed as a by-product in the cheese making process and whey protein are a combination of the proteins isolated from whey (generally, the proteins that are dissolved in or present in whey). In an embodiment, whey protein is a mixture of proteins and peptides that comprise α-lactalbumin, β-lactalbumin, serum albumin, various immunoglobulins, and glycomacropeptide. In an embodiment, whey may be isolated from mammalian milk that has been coagulated wherein the resulting byproduct of the non-coagulated portion comprises the whey. The whey can be derived from many of the mammalian animal species described herein. For example, the milk may be derived from primates, cows, sheep, pigs, goats, camels, horses, llamas, alpacas, and/or bison. Milk is coagulated by dropping the pH to an acid level that is approximately 4.5-4.7, or alternatively and/or additionally by adding enzymes (that separate the curd from the liquid whey). In an embodiment, whey protein is isolated from whey by pasteurizing the whey and concentrating the whey (by evaporation or for example, using a vacuum). The whey protein can be isolated either by membrane filtration and/or by chromatographic techniques such as ion exchange chromatography.

In an embodiment, whey protein may be further processed to generate a whey isolate that is free of or substantially free of lactose by further filtration. Alternatively and/or additionally, in an embodiment, whey or whey protein can be fermented by lactic acid bacteria. The lactic acid bacteria, in an embodiment, is Lactobacillus strain. In a variation, the whey, whey protein, or whey isolate, which may be fermented, may not just be edible but may also possess the added benefit of being able to treat diarrhea (for example, acute diarrhea) or other intestinal maladies, particularly, in infants who are malnourished.

An additional advantage to the whey-based microstructures of the present invention is its antibacterial properties (e.g., this may be a reason for the decrease in diarrhea). Thus, the use of whey-based microstructures may be used for its antibacterial properties to treat bacterial infections. Also, the use of these whey-based microstructures may also reduce the need for antibacterials in the cell growth process. Thus, these microstructures may eliminate the use of antibiotics in cell culture and make a clean cultivated product that requires fewer purification steps. The natural antibiotic properties of the whey-based microstructures may lead to a plurality of other benefits such as a lesser need for heating for long periods of time (for example, pasteurization) as an attempt to mitigate for the presence of bacteria. In a variation, because whey has many of the properties that are naturally present in mother's milk, it can be expected that the microstructures of the present invention will possess one or more of antimicrobial, antifungal, antiviral, immune-modulatory, and/or anti-carcinogenic (antioxidant) activities.

In an embodiment, the fermented whey-based microstructures can be used to expand cultures of various human or animal cells, including muscle cells, nerve cells, fat cells, and stem cells, such as embryonic stem cells, induced pluripotent stem cells, and adult stem cells. These cells may be sourced from humans or a variety of animals, including but not limited to other primates, cows, sheep, pigs, goats, camels, horses, llamas, alpacas, crocodiles, and bison. In a variation, the microstructures may also be used to produce cultivated products such as skin, leather, suede, wool, tissues, and organs. The use of fermented whey and whey derived from mammalian cells in the production of these microstructures may provide additional benefits such as being animal-free, improved protein solubility and increased antioxidant activity.

### The Microparticles

Some aspects of the present disclosure relate to compositions of dairy-based microstructures, such as microcarriers, scaffolds, and substrates, and methods of making or using these microstructures for cell culture and other applications. These microstructures may be made from milk proteins such as whey or casein proteins, which may be purchased from commercial vendors or extracted from milk products, mammalian cells or bacterial cultures. The dairy-based microstructures may include a plurality of microparticles or sheets made from milk proteins, which may be crosslinked (or alternatively, not crosslinked) using agents, such as transglutaminase, glutaraldehyde, 4,4′-diisothiocyano-2,2′-stilbenedisulfonic acid (DIDS), or 1,14-diazido-3,6,9,12-tetraoxatetradecane. In an embodiment, the microstructure comprises a plurality of sheets made from milk proteins, such as whey protein, which may be crosslinked (or alternatively, not crosslinked) using agents, such as transglutaminase, glutaraldehyde, or DIDS. In an embodiment, the sheets are characterized by their unique structural and mechanical properties, which make them ideally suitable for a variety of applications. For example, the crosslinked sheets may have improved mechanical strength and stability, making them ideal for use as scaffolds in tissue engineering or as microcarriers in cell culture. In a variation, the non-crosslinked sheets may have a more flexible and dynamic nature, making them suitable for use as soft substrates in cell culture or as materials for the production of cell-based meat products.

A combination of the crosslinked and non-crosslinked sheets may be used to give properties that are intermediate between the structural and mechanical properties of the crosslinked and the non-crosslinked sheets.

Overall, the dairy-based microstructure of the present disclosure represents a versatile and innovative tool for various applications in the field of cell culture and beyond. The crosslinking process may involve covalent bonding of protein molecules, resulting in a more stable, rigid, and mechanically strong microstructure. In some embodiments, the microparticles comprise whey proteins or proteins isolated from fermented whey and whey derived from mammalian cells that are not crosslinked. In embodiments, the microparticles or sheets may have specific mean diameters, areas, surface areas, thicknesses, and aspect ratios that are optimized for various applications. For example, microparticles with smaller diameters and higher surface areas may be more suitable for cell culture applications due to their increased surface area for cell attachment and their ability to provide more homogenous cell distribution. In some embodiments covalent and non-covalent coatings can be coupled with the outer structure of the dairy and non-dairy based microstructures. Non-covalent coatings can also involve layers of charged materials onto the outer surface, such as blood serum proteins of extracellular matrix proteins, antibodies and more. This can be composed of several layers of covalent and non-covalent binding. The animal or human cells can also be used to secrete other factors that can enhance the functionality of microcarriers. So that, the microcarriers can be precoated with cells or cell derivatives that express specific molecules or coatings needed for the microcarriers.

In some embodiments of the present invention, coatings may be applied to the surface of dairy-based or fermented dairy-based microstructures to modify their functional, physicochemical or biological properties. These coatings may be covalently and/or non-covalently coupled to the microstructures and may comprise a variety of components, including but not limited to:
a) Cell-binding ligands: These biomolecules bind to specific receptors on the surface of cells and promote their attachment and proliferation on the microstructures. Examples of cell-binding ligands include, but are not limited to, RGDS (arginine-glycine-aspartic acid-serine), which binds to the integrin receptors on many types of cells, and heparin-binding domains, which bind to the heparan sulfate proteoglycans on the cell surface.
b) Extracellular matrix products: These biomolecules are components of the extracellular matrix that provide structural support and signaling cues for cells. Examples of extracellular matrix products include, but are not limited to, proteoglycans (e.g., heparan sulfate, chondroitin sulfate, and keratan sulfate), non-proteoglycan polysaccharides (e.g., hyaluronic acid), and proteins (e.g., collagen, elastin, fibronectin, and laminin).
c) Blood serum ingredients or blood-related compounds. Blood serum includes but is not limited to all proteins that occur in blood that are not used in blood clotting (e.g., erythrocytes, leucocytes, and thrombocytes). Blood serum ingredients include but are not limited to electrolytes, antibodies, antigens, proteins, ions, sugars, signaling molecules, nutrients, lipids, fats, hormones (protein and non-protein chemicals), growth factors, salts, vitamins, exosomes and various exogenous substances that occur in blood.

In certain aspects of the present invention, a hydrated solution of whey protein may be processed to create a water-in-oil (w/o) emulsion. In a variation, the hydrated solution may be mixed with an oil phase and at least one surfactant or emulsifier. This combination may be homogenized to produce a w/o emulsion (water in oil emulsion) comprising whey protein microdroplets. The oil phase may be selected from a variety of oils, such as nonane, decane, undecane, n-dodecane, and the like, or mixtures thereof. The surfactant or emulsifier may be chosen from a range of options, including but not limited to sorbitan, stearate, polyglycerol oleate, lecithin, sorbitan monooleate, lanolin, polyglycerol polyricinoleate, or mixtures thereof. In some cases, the ratio of hydrated whey protein solution to emulsifier solution may be greater than or equal to 1:4, 1:5, 1:6, 1:7, 1:8, 1:9, 1:10, or 1:12. These w/o emulsions may be used in a variety of applications, including the production of dairy-based microstructures for cell culture and other purposes.

The present disclosure relates to the use of dairy-based microstructures for cell culture and other applications. These microstructures, which may be made from milk proteins such as whey or casein, can be used to culture a variety of human or animal cells, including stem cells and muscle, nerve, and fat cells. In certain embodiments, the dairy-based microstructures may be crosslinked using agents such as transglutaminase or glutaraldehyde, and may be coated with components such as cell-binding ligands or extracellular matrix products. The microstructures may be added to a bioreactor or other cell culture systems, where they can be used to expand a culture of cells or produce a cell cultivated product such as skin, leather, wool, tissues, or organs.

In some cases, the dairy-based microstructures may be sustainably harvested from milk-producing animals raised on regenerative farms. The present disclosure also relates to methods of making and using these microstructures, as well as kits involving them. Alternatively and/or additionally, the present disclosure includes the use of fermented whey and whey derived from mammalian cells in the creation of these microstructures, as well as the inclusion of flavor molecules found in meat to enhance the taste and nutritional value of the final product. Flavor molecules that are found in meat or other cell cultivated products include but are not limited to 2-methylpyrazine, hexanal, heptanal, isovaleraldehyde, hept-2-enal, nonanal, (E,E)-2,4-decdienal, octanal, 2-methyl-3-furanthiol, (2E)-2-undecanal, bis(2-methyl-3-furyl)disulfide, 2,3-diethyl-5-methylpyrazine, 2-octenal, 2,6-dimethylpyrazine, tridecanal, (2E)-2-nonenal, 1-octen-3-ol, 2-decenal, 2,4,5-trimethylthiazole, hexadecanal, 2-acetyl-1-pyrroline, 2-ethyl-5-methylpyrazine, 3-octen-2-one, furan-2-yl-methanethiol, 12 methyltridecanal, pentadecanal, methionol, myristyl aldehyde, 2-ethyl-3-methylpyrazine, mixtures thereof and dimethyl sulfide linkages.

To further improve the efficiency of cell culture, coatings may be created around the microstructures using cell-binding ligands or extracellular matrix products such as heparan sulfate, chondroitin sulfate, keratan sulfate, hyaluronic acid, collagen, elastin, fibronectin, or laminin. Cells within the microstructures may also be protected against shear stress in bioreactors using cell encapsulation methods. To increase the final density of cells grown on the edible or biodegradable microcarriers, various modifications may be made, including the optimization of culture conditions and the use of growth factors or other additives in the cell culture media.

### Flavor & Cell Density Enhancements Using the Edible Microcarriers:

In some embodiments, the dairy-based microstructures of the present disclosure may be modified to include flavor molecules found in meat or other cell cultivated products. This can be achieved by incorporating these molecules into the microparticles or sheets during or after the production process, for example by adding them to the protein solution before crosslinking or sheet formation. The use of whey or fermented whey and whey derived from mammalian cells as the starting material for these microstructures offers several advantages in this regard. First, both whey and fermented whey and whey derived from mammalian cells contain a wide range of bioactive compounds that can contribute to the flavor and functional properties of the final product. Second, these materials are highly versatile and can be modified using various techniques, such as fermentation, crosslinking, heating, and coating, to achieve the desired properties. Third, the use of whey or fermented whey and whey derived from mammalian cells as the starting material allows for production of these microstructures in a sustainable and cost-effective manner, as these materials are widely available and can be produced from milk by-products or through the fermentation of milk.

In an embodiment, dairy-based microparticles can be a fat replacer in meat production. In a variation, the dairy-based microparticles may be microparticulated whey protein. Microparticulated whey protein can be formed by thermal means and by shearing whey at low pH (4-5.5). This processing leads to the degradation of several of the whey proteins, and leads to exposing the cysteine moieties thiol groups. Subsequently, aggregation takes place gernating spheroid particles that are about 5 microns or less. The dairy or whey product may serve as a fat substitute and/or a fat mimetic. In a variation, the ideal is to replicate the physicochemical and sensorial properties of fat in food products. Whey protein has a similar flavor profile to fat and thus, it is not only highly compatible with dairy applications but it may be an ideal substance to be used in microcarriers. One benefit of using whey based products is that they have many of the sensory characteristics of fat without the concomitant level of calories present in animal fat. Thus, in one embodiment, the use of these dairy based microcarriers will be as a treatment for obesity. In a variation, the use of these products will give the consumer the sated feeling that occurs from high fat foods without the associated high calorie content.

Overall, the incorporation of flavor molecules found in meat or other cell-cultivated products into the dairy-based microstructures of the present disclosure represents a novel approach for the development of meat or other cell-based alternatives with improved sensory properties, such as texture, aroma, and taste, and other desirable functional characteristics.

Some of the desirable functional characteristics can be attained by the addition of the dairy based microcarriers of the instant invention because of their size, their viscosity (η), their firmness, their water holding capacity, their color, their coefficient of friction (μ), and other sensory properties. In an embodiment, the addition of whey protein makes a product that reduces firmness, restores some of the proteolytic activity, adds opaqueness and improves other sensory properties.

One approach to increase the cell density on these microstructures is to optimize the structural and mechanical properties of the microcarriers. For example, microparticles with smaller diameters and higher surface areas may be more suitable for cell culture applications due to their increased surface area for cell attachment and their ability to provide more homogenous cell distribution. Similarly, sheets with specific mean surface areas (200-15,000 cm²/gr) and thicknesses (10-500 um) may be optimized for cell attachment and growth. Additionally and/or alternatively, the use of crosslinked microstructures may also improve the mechanical strength and stability of the microcarriers, and by increasing stability, the crosslinked microstructures can support higher cell densities.

Another approach to increase the cell density on these microstructures is to modify the surface chemistry of the microcarriers. This can be achieved by coating the microstructures with molecules that promote cell adhesion and proliferation, such as cell-binding ligands, extracellular matrix products, and blood-related products. For example, the microstructures may be coated with RGDS, laminin, or fibrinogen, which can enhance cell attachment and proliferation on the microcarriers. In addition, the microstructures may be modified to release growth factors or other bioactive molecules that can stimulate cell growth and differentiation.

Finally, the growth conditions of the cells on the microstructures can also be optimized to increase the cell density. For example, the pH, temperature, oxygen level, carbon dioxide level, and nutrient levels in the culture medium can be adjusted to support optimal cell growth and differentiation. Sensors may be used to ascertain and/or maintain optimal levels. The sensors may be operationally attached to machines or other devices that allow the system of the invention to be autonomous (e.g., does not need input from a human). In addition, the culture medium may be supplemented with various growth factors, hormones, and other bioactive molecules that can stimulate cell growth and differentiation. For example, growth hormone or somatotropin may be added to the dairy-based microstructure containing mixture.

In an embodiment, the present disclosure provides several approaches for increasing the final cell density of cells grown on dairy-based microstructures, such as edible microcarriers, for use in static cell culture systems or bioreactors.

### Protection of the Cells Attached to the Edible Microcarriers Against Shear Stress in Bioreactor

In some embodiments of the present disclosure, biomaterial enclosures may be created around the final edible microcarriers with cells to protect against shear stress that the cells experience in bioreactors. These enclosures may be created using a variety of techniques or materials, including but not limited to:

### Microfluidic Techniques:

These techniques involve the use of microfluidic devices to precisely control the flow of fluids at the microscale and create droplets or capsules with defined sizes and shapes. These droplets or capsules can then be used as enclosures for the microcarriers with cells attached. Examples of microfluidic techniques that can be used include, but are not limited to, droplet-based microfluidics, microchannel-based microfluidics, and microfluidic interfacing with microelectromechanical systems (MEMS).

### Biopolymer-Based Techniques:

These techniques involve the use of biopolymers, such as alginate, chitosan, or gelatin, to create hydrogel-based enclosures for the microcarriers with cells attached. The biopolymers can be crosslinked using a variety of methods, including chemical crosslinking, physical crosslinking, or a combination of both.

### Other Techniques:

Other techniques that can be used to create biomaterial enclosures around the microcarriers with cells attached include, but are not limited to, electrospinning, printing, bioprinting, self-assembly, and template-assisted methods.

These biomaterial enclosures may provide a protective environment for the cells, shielding them from shear stress and other mechanical forces that they may experience in bioreactors. This may help to improve the viability and functionality of the cells, ultimately leading to increased final cell densities on the edible microcarriers.

### Examples

For the below protocols 1 and 2, the preparation of whey microparticles includes both the preparation of non-crosslinked and/or crosslinked whey microparticles. All of the protocols enumerated below can be modified with proteins from fermented whey and whey derived from mammalian cells or a mix of fermented whey proteins and non-fermented whey proteins. Whey proteins include but are not limited to β-lactoglobulin (β-LG, for short), α-lactalbumin (α-LA), immunoglobulins (IG), bovine serum albumin (BSA), bovine lactoferrin (BLF) glycomacropeptides, lactoferrins, and lactoperoxidases (LP).

### Protocol 1

In some embodiments of the present disclosure, a method of preparing dairy-based microstructures for use in cell culture and other applications is disclosed. The method involves dissolving 5% (w/v) whey protein in phosphate buffered saline, stirring the solution at a speed of 1000-1500 rpm for a certain amount of time (˜5 min), and filtering it through a hydrophilic syringe filter. The filtered solution is then left at room temperature to complete the hydration process. Separately, a solution of 2% (w/v) polyglycerol polyricinoleate (PGPR) is prepared in n-dodecane and stirred for a specific period of time (˜5 min) at room temperature. The whey protein solution is then added to the PGPR solution and the resulting water-in-oil (w/o) emulsion is homogenized using a high-speed mixer for 15 min. The size of the whey protein droplets in the emulsion is varied by adjusting the time and mixing speed. The w/o emulsion is then placed in sealed glass tubes and heated to 80° C. for 15 min to form stable microparticles. The microparticles are then cooled and separated from the w/o emulsion, rinsed to remove non-gelled material, and redispersed in phosphate buffered saline. The microparticles can then be split into two batches, with one batch being used as is and the other batch treated with 2.5% (w/v) transglutaminase to crosslink the microparticles.

### Protocol 2

Some aspects of the present disclosure relate to a method of producing dairy-based microstructures for use in cell culture and other applications. In some embodiments, the method comprises dissolving 5% (w/v) whey protein in a phosphate buffered saline solution and stirring the solution for 5 min to allow for the complete hydration of the whey protein. The solution is then filtered using a hydrophilic syringe filter and left at 4° C. overnight.

The hydrated whey protein solution can then be used to produce either non-crosslinked or crosslinked dairy-based microstructures. To produce non-crosslinked microstructures, a sodium chloride solution is added to the whey protein solution, causing the dissolved whey protein to form microparticles through the process of salting out. Alternatively, crosslinked microstructures can be produced by adding transglutaminase to the whey protein solution, which crosslinks the dissolved whey protein into microparticles.

The resulting microparticles are separated from the solution and rinsed multiple times to remove any non-gelled material. The microparticles can then be redispersed in phosphate buffered saline or dried and stored until use. In some embodiments, the dairy-based microstructures produced using this method may have a mean diameter of greater than or equal to 60 micron, and a mean area of greater than or equal to 1200 square micron. The microstructures can be used for a variety of applications, including expanding a culture of human or animal cells or producing a cultivated product.

### Protocol 3: Flavor Enhanced Edible Microcarriers

To produce dairy-based microstructures enhanced with flavor molecules and custom proteins, the following protocol may be followed:

Obtain a purified source of whey protein, such as by purchasing from a commercial vendor or extracting directly from milk. Dissolve the whey protein in a phosphate buffered saline solution at a final concentration of 150 g/kg. Stir the solution at room temperature for 2 hours, then filter through a 0.45-micron hydrophilic syringe filter. Add flavor molecules and custom proteins to the filtered whey protein solution, using a ratio appropriate for the desired final concentrations of these additives.

Heat the solution to 80° C. for 15 minutes to denature the proteins and facilitate the incorporation of the flavor molecules and custom proteins. Rapidly cool the solution to room temperature, then adjust the pH to 7.0. Add sodium chloride or transglutaminase to the solution, as desired, to produce non-crosslinked or crosslinked microparticles, respectively. Separate the microparticles from the solution and rinse thoroughly to remove any non-gelled material. Redisperse the microparticles in a phosphate buffered saline solution or dried them, then store until use.

### Protocol 4

To enhance the final cell density of cells grown on these dairy-based microstructures, the following modifications may be made to the above protocols:

The concentration of whey protein in the solution can be optimized to maximize cell attachment and proliferation. The microstructures can be coated with cell-binding ligands or extracellular matrix components, such as heparan sulfate, chondroitin sulfate, keratan sulfate, hyaluronic acid, collagen, elastin, fibronectin, laminin, etc. The cells can be cultured in an animal-free media made from recombinant proteins and growth factors. Bioreactors can be used that reduce shear stress on cells. Alternatively and/or additionally, other cell culture systems can be used that minimize shear stress on the cells. Techniques that act to reduce shear stress include but are not limited to gentle stirring or perfusion methods. The cell density can be monitored by sensors and the viability of the cells can also be monitored and/or ascertained throughout the culture period, and the culture conditions can be adjusted as needed to maximize the final cell density.

### Protocol 5

To protect the cells against shear stress in bioreactors, the following modifications may be made to the above protocols:

In an embodiment, biomaterial enclosures can be created around the final edible microcarriers with cells attached to protect against shear stress that cells typically experience in a bioreactor. To create the biomaterial enclosures, a variety of materials may be used, including but not limited to, synthetic polymers (e.g., polyethylene, polypropylene, polystyrene, polyethylene terephthalate, etc.), natural polymers (e.g., collagen, elastin, chitosan, etc.), and composite materials (e.g., polyethylene terephthalate coated with collagen, polystyrene coated with chitosan, etc.). These biomaterial enclosures provide a barrier that reduces the amount of shear stress that the cells that are growing will experience.

In order to enhance the whey proteins and microparticles with flavor molecules and custom proteins, the following modifications may be made to the above protocols:

Flavor molecules may be obtained from a natural source, such as meat, vegetables, fruit, or other natural resources. These flavor molecules can be extracted using a variety of methods, including but not limited to solvent extraction, supercritical fluid extraction, and enzyme-assisted extraction.

The extracted flavor molecules can be incorporated into the whey protein solution by adding the flavor molecules to the solution at a predetermined concentration. Any combination of the flavor molecules may be added and the concentrations can be adjusted to adjust the flavor.

Custom proteins can be created by expressing the desired proteins in a host organism, using procaryotic or eukaryotic cells, such as bacteria or yeast, using recombinant DNA technology. These custom proteins can be incorporated into the whey protein solution in the same manner as the flavor molecules. Alternatively and/or additionally, the proteins may be separated and/or isolated from natural sources using the extraction or separation techniques described herein.

To enhance the final cell density of cells grown on the edible microcarriers, the following modifications may be made to the above protocols.

The composition of the cell culture growth media can be optimized by selecting appropriate animal-free media created from recombinant proteins, as well as using appropriate concentrations of growth factors and other supplements.

In an embodiment, the surface properties of microcarriers may be modified by coating the surface with cell-binding ligands or extracellular matrix proteins, such as heparan sulfate, chondroitin sulfate, keratan sulfate, hyaluronic acid, collagen, elastin, fibronectin, and laminin.

In an embodiment, the mechanical properties of the microcarriers can be controlled by the addition of other edible or biodegradable proteins. In an embodiment, the present invention contemplates the use of plant-based proteins, such as Triticeae (wheat) derived proteins to alter the mechanical properties of the microcarriers and to add porosity to the microcarriers. In a variation, Triticum aestivum or Triticum spelta can be used. In a variation, Triticeae gluten can be used. In a variation, the one or more Triticeae-derived proteins can be mixed with proteins that are derived from other sources other than the Triticeae genus. In an embodiment, a soy derived product may be used. In a variation, textured soy protein may be used.

In an embodiment, the present invention relates to a dairy-based microstructure for use as a microcarrier in a cell culture system, the dairy-based microstructure comprising: a micron-sized particle of dairy-based material; and a surface coating configured to support attachment and growth of adherent cells. The surface coating is as described herein and is applied on the surface of the dairy-based microcarrier. In a variation, the micron sized particle is between about 50 and 1000 micrometers in diameter. In a variation, the micron-sized bead is greater than about 60 and less than 1000 micrometers. In a variation, the micron-sized bead is less than 50 micrometers. In a variation, the micron-sized bead is above 1000 micrometers.

In a variation, the microstructure may be further configured for use as a scaffold or substrate in a cell culture system. In a variation, the dairy-based microstructure has a porosity level that allows cells to be cultured at a higher surface area per volume relative to a planar culture. In a variation, the porosity level is such that at least about 25% of the volume of the dairy-based microstructure is void volume. In a variation, the porosity level is such that at least about 30% of the volume of the dairy-based microstructure is void volume. In a variation, the porosity level is such that at least about 35% of the volume of the dairy-based microstructure is void volume. In a variation, the porosity level is such that at least about 40% of the volume of the dairy-based microstructure is void volume. In a variation, the porosity level is such that at least about 55% of the volume of the dairy-based microstructure is void volume. In a variation, the void volume is present as pores and the diameters of the void volume pores is between about 2 pm to 1.5 mm, or from 10 pm to 250 μm, or from 25 pm to 100 μm.

In a variation, the surface coating comprises cell-binding ligands, extracellular matrix proteins, or blood serum products. In a variation, the cell-binding ligands or extracellular matrix proteins are one or more members selected from but not limited to the group consisting of heparan sulfate, chondroitin sulfate, keratan sulfate, hyaluronic acid, collagen, elastin, fibronectin, and laminin.

In an embodiment, the present invention relates to a method of making a dairy-based microstructure for use as a microcarrier in a cell culture system, the method comprising: providing a micron-sized particle of a dairy-based material; and applying a surface coating to the particle configured to support the attachment and growth of adherent cells. The surface coating may be comprised of the materials discussed herein.

In a variation of the method, the method may further comprise configuring the microstructure for use as a scaffold or substrate in a cell culture system.

In a variation, the method may further comprise providing a porosity level to the microstructure that allow cells operationally attached to the micron-sized bead to be cultured at a higher surface area per volume relative to cells grown in a planar culture.

In an embodiment, the present invention relates to a method of using a dairy-based microstructure for cell culture, the method comprising: providing a dairy-based microstructure comprising a micron-sized bead of dairy-based material and a surface coating configured to support the attachment and growth of adherent cells; and introducing the adherent cells to the surface of the dairy-based microstructure in a cell culture system.

In a variation, the method may further comprise using the microstructure as a scaffold or a substrate in the cell culture system. The cell culture system may be present in a bioreactor or a vertical cellular farm.

In an embodiment, the present invention relates to a dairy-based microstructure for use as an edible microcarrier in a cell culture system. In a variation, the edible microcarrier may be used in the cultivation of cell-based meat or other cell-based applications. In a variation, the microstructure may comprise: a micron-sized bead of dairy-based material and a surface coating configured to support attachment and growth of cells in the cell culture system.

In a variation, the dairy-based microstructure may further comprise a porosity level that allows cells to be cultured at a higher surface area per volume relative to cells grown in a planar culture.

In an embodiment, the present invention relates to a method of using a dairy-based microstructure for the cultivation of cell-based meat or other cell-based applications, the method comprising: providing a dairy-based microstructure comprising a micron-sized bead of dairy-based material and a surface coating configured to support the attachment and growth of cells.

In a variation, the method may further comprise using the microstructure as a scaffold or substrate in the cell culture system for the cultivation of cell-based meat. In a variation, the cell culture system can be used for other cell-based applications.

In an embodiment, the present invention relates to a system for the large scale cultivation of cell-based meat or other cell-based applications, wherein said system comprises: a bioreactor; a dairy-based microstructure comprising a micron-sized bead of dairy-based material, a surface coating configured to support attachment and growth of cells; and cells introduced on to a surface of the dairy-based microstructure in a bioreactor for the large scale cultivation of cell-based products. In a variation, large scale cultivation of cell-based products is of a scale that fills a bioreactor. In a variation, a bioreactor is 50 liters or smaller. In a variation, a bioreactor is 100 liters. In a variation, a bioreactor is 250 liters. In a variation, a bioreactor is 500 liters. In a variation, a bioreactor is 1000 liters. In a variation, a bioreactor is 2000 liters or larger. In a variation, the cell-based products may be grown at levels wherein the cells in the bioreactor are present at 10⁵ cells/ml or less than that. In a variation, the cell-based products may be grown at levels wherein the cells are present at 10⁶ cells/ml. In a variation, the cell-based meat may be grown at levels wherein the cells are present at 10⁷ cells/ml or above that.

In a variation, in the system, the microstructure may be configured for use as a scaffold or substrate in the bioreactor for the cultivation of cell-based meat, or generally, for other cell-based applications.

In an embodiment, the present invention relates to a dairy-based microstructure for use as a microcarrier in cell culture, wherein the microstructure comprises: a dairy-based matrix; and at least one protein or flavor molecule incorporated into the dairy-based microstructure.

In a variation, the dairy-based microstructure comprises a protein that is a custom protein selected to provide a desired flavor or texture to the cell-based products using the microstructure. Custom protein(s) that may be added include one or more of soy or soybean protein, brown rice protein, sacha inchi protein, caseinate protein, pea protein, proteins from nuts, proteins from berries, heme proteins, wheat protein, and/or potato protein. Other proteins that may be added include the proteins that appear in blood serum. A desired flavor can often be achieved by combining one or more of the proteins disclosed herein with one or more of the flavor molecules disclosed herein. Because flavor is enhanced by the aroma (i.e., the two are linked), the flavor molecules and proteins that should be added are often the combinations that give good and/or pleasant aroma(s). Esters and terpene products are two types of chemicals that are known to give generally pleasant aromas to most people. Often times the protein component may not be added to give flavor and/or smell but rather to enhance the aroma/taste of the product that contains flavor molecules.

In a variation, the flavor molecule is a natural plant extract or a synthetic flavor compound. In a variation, the synthetic flavor compound is made and/or modified from extracts from plants. In a variation, the dairy-based microstructure may further act as or comprise a scaffold or substrate for supporting cell growth. The flavor molecule may be one or more of peptides, vitamins, minerals, yeast extracts, diacetyl, ethyl decadienoate, ethyl maltol, ethyl propionate, ethyl butanoate, octyl acetate, ethyl heptanoate, pentyl butanoate, and other esters, ethylvanillin, eucapyptol, isoamyl acetate, 2,6-lutidine, limonene, manzanate, menthone, menthol, benzyl alcohol, ethyl maltol, furaneol, benzaldehyde, hexanal, cinnamaldehyde, citral, neral, nerol, vanillin, methyl anthranilate, glutamic acid salts, glycine salts, guanylic acid salts, inosinic acid salts, 5′-ribonucleotide salts, monosodium glutamate, indoles, putrescine, trimethylamine, octenone, acetyl pyrroline, acetyl tetrahydropyridine, lactones, terpenes, sweeteners such as glucose and fructose, saccharin, cyclamates, aspartame, and mixtures thereof.

In an embodiment, the present invention relates to a method of creating a dairy-based microstructure, the method comprising: combining a dairy-based matrix with at least one protein or flavor molecule to form a mixture; and shaping the mixture into the dairy-based microstructure as described in the above examples. The dairy-based matrix may comprise extracellular matrix products as described herein. In a variation, the method may further comprise a step of sterilizing the microstructure prior to use. In a variation of the method, the method may further comprise a step of incorporating the microstructure into an animal-free media formulation for use in a cell culture system.

In an embodiment, the present invention relates to a method of using a dairy-based microstructure to produce cultivated meat or other cell-based products in a bioreactor, the method comprising: introducing the dairy-based microstructure into a bioreactor containing a serum-free media formulation of recombinant proteins and growth factors; and culturing the microstructure to produce cultivated meat, or alternatively producing other cell-based products.

In a variation, the method may further comprise a step of modifying the microstructure with at least one protein or flavor molecule to enhance the flavor of the cultivated meat, or other cell-based products.

In an embodiment, the present invention relates to a system for producing cultivated meat or other cell-based products, wherein the system comprises: a bioreactor containing an animal-free media formulation of recombinant proteins and growth factors; a dairy-based microstructure introduced into the bioreactor, the microstructure comprising a dairy-based matrix; and at least one protein or flavor molecule incorporated into the dairy-based microstructure.

In an embodiment, the present invention relates to a cell culture system comprising: a dairy-based microstructure, wherein the dairy based microstructure comprises a micron-sized particle of dairy based material, adherent cells, and a surface coating configured to support attachment and growth of the adherent cells. In a variation, the surface coating is on a surface of the micron-sized particle.

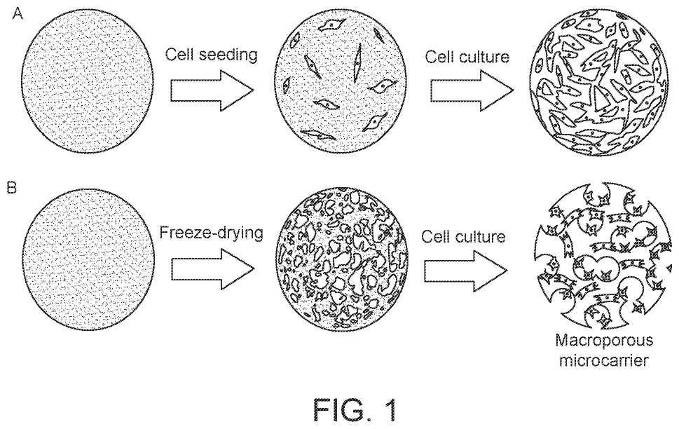

FIG. 1 shows exemplary drawings of cell seeding (A) and freeze drying (B) to make cell cultures using the microstructures of the present invention. The cells proliferate on spherical and solid microcarriers in FIG. 1 (A), while they can penetrate inside microporous microcarriers in FIG. 1 (B) and form a 3D cell-laden construct.

### The Following References are Incorporated by Reference for all Purposes:

Applications of Biotechnology to Fermented Foods: Report of an Ad Hoc Panel of the Board on Science and Technology for International Development, 1992, National Academies Press, National Research Council.
Review on fat replacement using protein-based microparticulated powders or microgels: A textural perspective, Kew, Ben et al., Trends Food Sci. Techol. 2020 Dec., 106, 457-468.
Whey proteins and their antimicrobial properties in donkey milk: a brief review, Brumini, Diana et al., Dairy Science & Technology, 2016, 96, 1-14.
Multifunctional Thermoresponsive Microcarriers for High-Throughput Cell Culture and Enzyme-Free Cell Harvesting, Dabiri et al., Small, 2021, 17, 2103192.
Stiffness-Controlled Thermoresponsive Hydrogels for Cell Harvesting with Sustained Mechanical Memory, Fan et al., Adv. Healthcare Mater., 2017, 6, 1601152.
Enzyme-Free Cell Detachment Mediated by Resonance Vibration With Temperature Modulation, Kurashina et al., Biotechnology and Bioengineering, 114(10), 2017, 2279.
Whey Proteins Cross-linked by Transglutaminase or Glycated with Maltodextrin: Physicochemical Bases of the Improved Heat Stability, Wan Wang Dissertation, University of Tennessee, Knoxville, 2013.
Enzymatic cross-linking of proteins with tyrosinase, Thlmannn et al., Eur Food Res Technol, 214, 2002, 276.
Reducing the stiffness of concentrated whey protein isolate (WPI) gels by using WPI microparticles, Purwanti et al., Food Hydrocolloids 26, 2012, 240.
Modification of fermented whey protein concentrates: Impact of sequential ultrasound and TGase cross-linking, Gantumur et al., Food Research International, 163, 2023, 112158.
Mechanical ageing of whey protein-based fouling layers by temperature-dependent gradual solidification, Schnöing et al., Food and Bioproducts Processing, 133, 2022, 67.
Citric acid promotes disulfide bond formation of whey protein isolate in non-acidic aqueous system, Li et al., Food Chemistry, 338, 2021, 127819.
Batch versus microfluidic emulsification processes to produce whey protein microgel beads from thermal or acidic gelation, Lacroix et al., Journal of Food Engineering, 312, 2022, 110738.
Crosslinking of Whey Protein by Transglutaminase, Aboumahmoud et al., J Dairy Sci., 73, 1989, 256.
Whey protein microgel particles as stabilizers of waxy corn starch+locust bean gum water-in-water emulsions. Murray et al., Food Hydrocolloids, 56, 2016, 161.
Acid gelation of whey protein microbeads of different sizes. Andoyo et al., Dairy Sci. & Technol., 96, 2016, 213.
Effect of crosslink density on the water-binding capacity of whey protein microparticles. Peters et al., Food Hydrocolloids, 44, 2015, 277.
Whey protein particles modulate mechanical properties of gels at high protein concentrations, Sa\check{g}lam et al., Food Hydrocolloids, 38, 2014, 163.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A dairy-based microstructure for use as a microcarrier in a cell culture system, the dairy-based microstructure comprising: a micron-sized particle of dairy-based material; and a surface coating configured to support attachment and growth of adherent cells.

2. The dairy-based microstructure of claim 1, wherein the dairy-based microstructure is further configured for use as a scaffold or substrate in a cell culture system.

3. The dairy-based microstructure of claim 1, wherein the dairy-based microstructure has a porosity level that allows cells to be cultured at a higher surface area per volume relative to a planar culture.

4. The dairy-based microstructure of claim 1, wherein the surface coating comprises cell-binding ligands, extracellular matrix proteins, or blood serum products.

5. The dairy-based microstructure of claim 4, wherein the cell-binding ligands or extracellular matrix proteins comprise one or more members selected from but not limited to the group consisting of heparan sulfate, chondroitin sulfate, keratan sulfate, hyaluronic acid, collagen, elastin, fibronectin, and laminin.

6. A method of making a dairy-based microstructure for use as a microcarrier in a cell culture system, the method comprising: providing a micron-sized particle of dairy-based material; and applying a surface coating to the micron-sized particle configured to support attachment and growth of adherent cells.

7. The method of claim 6, further comprising configuring the dairy-based microstructure for use as a scaffold or substrate in a cell culture system.

8. The method of claim 6, further comprising providing a porosity level to the dairy-based microstructure that allow cells to be cultured at a higher surface area per volume level relative to cells cultured in a planar culture.

9. A method of using a dairy-based microstructure for cell culture, the method comprising:
providing the dairy-based microstructure that comprises a micron-sized particle of dairy-based material and a surface coating configured to support attachment and growth of adherent cells; and introducing cells to a surface of the dairy-based microstructure in a cell culture system.

10. The method of claim 9, further comprising using the dairy-based microstructure as a scaffold or substrate in the cell culture system, wherein the cell culture system is one or more of a bioreactor or any cell cultivation system.

11. A dairy-based microstructure for use as an edible or biodegradable microcarrier in a cell culture system for the cultivation of cell-based meat or other cell-based applications, the dairy-based microstructure comprising: a micron-sized particle of dairy-based material;
and a surface coating configured to support attachment and growth of cells.

12. The dairy-based microstructure of claim 11, further comprising a porosity level that allows cells to be cultured at a higher surface area per volume relative to cells cultured in a planar culture.

13. A method of growing cell-based meat or one or more other cell-based compounds, the method comprising procuring a dairy-based microstructure for cultivation of the cell-based meat or one or more other cell-based compounds, providing a dairy-based microstructure comprising a micron-sized particle of dairy-based material and a surface coating configured to support the attachment and growth of cells to produce the cell-based meat or the one or more other cell-based compounds.

14. The method of claim 13, further comprising using the dairy-based microstructure as a scaffold or substrate for the cultivation of cell-based meat or the one or more other cell-based compounds.

15. A system for large scale cultivation of cell-based meat or other cell-based compounds, said system comprising:
a bioreactor; a dairy-based microstructure comprising a micron-sized particle of dairy-based material; a surface coating configured to support attachment and growth of cells;
and cells introduced to a surface of the dairy-based microstructure in the bioreactor for the large scale cultivation of cell-based meat or other cell-based compounds.

16. The system of claim 15, wherein the dairy-based microstructure is configured for use as a scaffold or substrate in the bioreactor for the large scale cultivation of the cell-based meat or the other cell-based compounds.

17. A dairy-based microstructure for use as a microcarrier in cell culture, the dairy-based microstructure comprising: a dairy-based matrix; and at least one protein or flavor molecule incorporated into the dairy-based microstructure.

18. The dairy-based microstructure of claim 17, wherein the at least one protein is a custom protein selected to provide a desired flavor or texture to the cell-based products using the dairy-based microstructure.

19. The dairy-based microstructure of claim 17, wherein the flavor molecule is a natural plant extract or synthetic flavor compound.

20. The dairy-based microstructure of claim 17, wherein the dairy-based microstructure further comprises a scaffold or substrate for supporting cell growth.

21. A method of creating a dairy-based microstructure, the method comprising: combining a dairy-based matrix with at least one protein or flavor molecule to form a mixture; and
then making dairy-based microstructure.

22. The method of claim 9, further comprising a step of sterilizing the dairy-based microstructure before use.

23. The method of claim 9, further comprising the step of incorporating the microstructure into an animal-free media formulation for use in a cell culture system.

24. A method of using a dairy-based microstructure to produce cultivated meat or one or more other cell-based compounds in a bioreactor, comprising: introducing the dairy-based microstructure into the bioreactor, wherein said bioreactor comprises a serum-free media formulation of recombinant proteins and growth factors; and culturing the dairy-based microstructure to produce the cultivated meat or the one or more other cell-based compounds.

25. The method of claim 24, further comprising a step of modifying the dairy-based microstructure with at least one protein or flavor molecule to enhance a flavor of the cultivated meat or the one or more other cell-based compounds.

26. A system for producing cultivated meat or other cell-based compounds, wherein said system comprises: a bioreactor containing an animal-free media formulation of recombinant proteins and growth factors; and a dairy-based microstructure introduced into the bioreactor, the dairy-based microstructure comprising: a dairy-based matrix and at least one protein or flavor molecule incorporated into the dairy-based microstructure.

27. A cell culture system comprising:
a dairy-based microstructure, wherein the dairy based microstructure comprises a micron-sized particle of dairy based material,
adherent cells, and a surface coating configured to support attachment and growth of the adherent cells.

28. The cell culture system of claim 27, wherein the surface coating is on a surface of the micron-sized particle.

29. The cell culture system of claim 27, wherein the dairy-based microstructure comprises one or more fermented whey and whey derived from mammalian cells products.
